(19) United States
(12) Patent Application Publication
(10) Pub. No.: US 2023/0384307 A1
(43) Pub. Date: Nov. 30, 2023

(54) IMPROVED DETECTION OF LYMPHOCYTE - TARGET CELL INTERACTION

(71) Applicant: LUMICKS CA HOLDING B.V., Amsterdam (NL)

(72) Inventors: Andrea CANDELLI, Amsterdam (NL); Keith Alan BAILEY, Amsterdam (NL)

(21) Appl. No.: 18/032,597

(22) PCT Filed: Oct. 20, 2021
(86) PCT No.: PCT/NL2021/050637

§ 371 Date: Apr. 19, 2023

(30) Foreign Application Priority Data
Oct. 20, 2020 (NL) 2026714

(51) Int. Cl. G01N 33/569; G01N 15/14; G01N 29/22

(52) U.S. Cl. CPC G01N 33/56972; G01N 15/1436; G01N 29/22

(57) ABSTRACT

The current invention relates to an improved method for detecting and/or measuring interaction between lymphocytes and target cells. In particular the current invention relates to a method for improved differentiation between lymphocytes and target cells that have formed (mature) immunological synapses and lymphocytes and target cells that have not, or only to a limited extend, formed such immunological synapses. The current invention can be used to discriminate between lymphocytes and target cells that specifically interact with each other, for example when the lymphocyte specifically interacts with an antigen presented on the target cell, and those that do not or only to a limited extend.

### TECHNICAL FIELD

The current invention relates to an improved method for detecting and/or measuring interaction between lymphocytes and target cells. In particular the current invention relates to a method for improved differentiation between lymphocytes and target cells that have formed (mature) immunological synapses and lymphocytes and target cells that have not, or only to a limited extend, formed such immunological synapses. The current invention can be used to discriminate between lymphocytes and target cells that specifically interact with each other, for example when the lymphocyte specifically interacts with an antigen presented on the target cell, and those that do not or only to a limited extend.

### BACKGROUND OF THE INVENTION

The description of the background of the invention includes information that may be useful in understanding the present invention. It is not an admission that any of the information provided herein is prior art or relevant to the presently claimed invention, or that any publication specifically or implicitly referenced is prior art.

A synapse is a specialized structure that forms when the plasma membranes of two cells come into close apposition to transmit signals. Well-known examples are the stable structures formed between two neurons, or between neurons and other cell types such as muscle cells. However, cells of the immune system also form synapses that, although more transient than neural synapses, are essential for cell activation and function. Lymphocytes such as T cells, B cells and natural killer (NK) cells form synapses that are referred to as immunological synapses. The immunological synapse typically forms between immune cells and antigen presenting cells, such as in the case of B and T helper cells, or between the immune cell and a target cell for killing, like in the case of cytotoxic T cells or natural killer cells.

In the case of T cells, the immunological synapses are formed between the lymphocytes and antigen-presenting cells (APCs) during the recognition of the peptide antigen-major histocompatibility complex (pMHC) ligand by the T-cell antigen receptor (TCR). The TCR and pMHC are both membrane-bound so it is clear that the TCR will only be triggered by its ligand at the interface between T cells and APCs.

Immunological synapses were first observed on the T cell-APC interface as two rings by confocal microscopy and these rings were named the central supramolecular activation cluster (cSMAC) and peripheral supramolecular activation cluster (pSMAC). The TCR has been reported to be present in the cSMAC, whereas other lymphocyte specific proteins such as lymphocyte function-associated antigen-1 (LFA-1), are integrated into the pSMAC ring that surrounds the TCR. The formation of this two-ringed structure (“bull's eye”) is however not universal and other formations such as “multifocal immunological synapses” between T cells and dendritic cells have been described. Therefor the immunological synapse is now broadly considered to be any structure formed at the interface of functional lymphocyte-target cell contact, such as for example T-cell-APC contacts (Alarcon et al. Immunology. 2011 August; 133(4): 420-425. doi: 10.1111/j.1365-2567.2011.03458.x).

One of the roles of the immunological synapse formation is to stop a lymphocyte from moving around in search of, in the case of for example T cells, an antigen to bind to. Indeed, lymphocytes are constantly moving through the body in search of antigen. When they finally recognize antigen, an important step is that they must stop and form a stable contact with the antigen-presenting cell. The antigen-induced up-regulation of LFA-1 (expressed by the lymphocyte) binding to ICAM-1 (expressed by the target cell presenting the antigen) to generate a strong adhesive surface is a critical step in slowing cell motility (Dustin et al. Cold Spring Harb Perspect Biol. 2010 October; 2(10): a002311. doi: 10.1101/cshperspect.a002311).

Indeed, it is believed that T cells are activated when TCRs and the co-receptors bind their respective ligands on the surface of APCs. This initial ligand-receptor recognition leads to the formation of the immunological synapse. It is believed that as T cell signaling proceeds, ligand-bound membrane receptors and signaling proteins, such as TCRs, co-stimulatory receptors and adhesion molecules, are clustered and reorganized in the synapse to form a pattern of several distinct concentric domains.

In other words, the formation of immunological synapses between a lymphocyte and a target cell, for example an APC, is a hall-mark event and signals the presence of specific interactions (i.e. the specific interaction between, for example, a TCR and an antigen recognized by the TCR) between the lymphocyte and the target cell that are involved in the formation of such immunological synapses.

As already mentioned above one of the roles of the immunological synapse formation is to stop a lymphocyte from moving around. Indeed, immunological synapses formed between lymphocytes and target cells are characterized by high rupture forces. Using biophysical measurements Hosseini (Hosseini et al. PNAS 2009 106:42 17852-17857 doi“10.1073”pnas.0905384106) showed that interaction forces between T cells and APCs develop over time and are highest when immunological synapse formation is maximal. They concluded that it is likely that strong interaction forces will favor optimal T-cell activation. Indeed, it was suggested that the potency of cellular immune responses strongly depends on T cell avidity to antigen (loannidou et al. Scientific Reports volume 7, Article number: 44320 (2017)). Others have made similar claims and have proposed improved methods for determining cell-cell rupture forces. For example, WO 2018/083193 discloses a method, system and sample holder for manipulating and/or investigating cellular bodies; It makes use of acoustic forces generated by ultrasound standing waves in a microfluidic flow-cell and is described in more detail with respect to FIG. 2-5 below. The acoustic force allows application of force to thousands of cells in parallel. For example, to thousands of T-cells in contact with a monolayer of target cells. By detecting cell-cell rupture events (e.g. T-cells releasing from a tumor cell monolayer) as a function of the acoustic force and/or by application of fluid flow in relation to an applied acoustic force, cell-cell binding interactions may be efficiently analyzed and/or cells may be sorted according to cell-cell binding interactions. For example, an avidity curve can be generated by plotting the percentage of bound CAR T-cells as a function of the applied force.

It is therefore not surprising that there is an interest in the field in methods that would allow to more precisely study the interaction between lymphocytes and their target cells, in methods that would allow better differentiation between lymphocytes and target cells that specifically bind with each other, and those that do not, or in methods that would allow to sort cells based on their binding interaction with each other, for example based on the interaction force between lymphocytes and target cells.

Although it appears that at least some of the techniques available in the prior art suffer from low signal-to-noise ratio, slow detection, and/or small sample size.

In light of this, improved methods that would allow for meeting the above-mentioned needs would be highly desirable but are not yet readily available. In particular, there is a clear need in the art for reliable, efficient and reproducible methods that allow the better study of the (specific) interaction between lymphocytes and their target cells. Accordingly, the technical problem underlying the present invention can be seen in the provision of such methods for complying with any of the aforementioned needs. The technical problem is solved by the embodiments characterized in the claims and herein below.

### DESCRIPTION OF THE INVENTION

### Drawings

Embodiments of the invention are further described hereinafter with reference to the accompanying drawings.

FIG. 1: Effect of an antagonist of LFA-1, BIRT-377, on CAR T cell binding to target cells expressing a defined antigen. A. Lines depict percentage of indicated bound cells to the immobilized target cells (monolayer), either or not in the presence of BIRT-377 (BIRT, 10 ug/mL), in relation to a relative force rForce that was applied to the interacting cells (the relative force is the average force over the region of interest experienced by 10.1 polystyrene beads as determined in the calibration procedure described with reference to FIG. 5 below. The medium used for the calibration was PBS with 0.02% pluronics, and 0.02% casein. Because of the difference in size, density and compressibility of the CAR T cells the true average force experienced by the CAR T cells is estimated to be about 3 times lower). Non-transduced peripheral blood mononuclear cells (PBMC) were used as controls. CAR T (neg) are primary human T cells expressing a non-specific CAR; CAR T (pos) are primary human T cells expressing a specific CAR (to the antigen expressed by the target cells). B. Bars show the percentage of human primary T cells expressing a non-specific (CAR T (neg, grey bars) or a specific CAR (CAR T (pos), black bars), or PBMC controls (white bar), bound to monolayer target cells at 500 pN of relative force as a function of the BIRT incubation time. Dotted lines indicate window of bound T cells (%) between lowest and highest binders.

FIG. 2: is a schematic drawing of an embodiment of an acoustic force manipulation system 1 which may be used in accordance with the present concepts.

FIG. 3: is a cross section of a sample holder and FIG. 3A is a detail of the sample holder of FIG. 3 as indicated with “IIA”.

The system 1 comprises a sample holder 3 comprising a holding space 5 for holding a sample 7 comprising one or more biological cells of a second type 9 in a fluid medium 11. The fluid preferably is a liquid or a gel. The system 1 may further comprise an acoustic wave generator 13, e.g. a piezo element, connected with the sample holder 3 to generate an acoustic wave in the holding space 5 exerting a force on the cells of a second type 9 in the sample 7. The acoustic wave generator 13 is connected with an optional controller 14 and power supply, here being integrated.

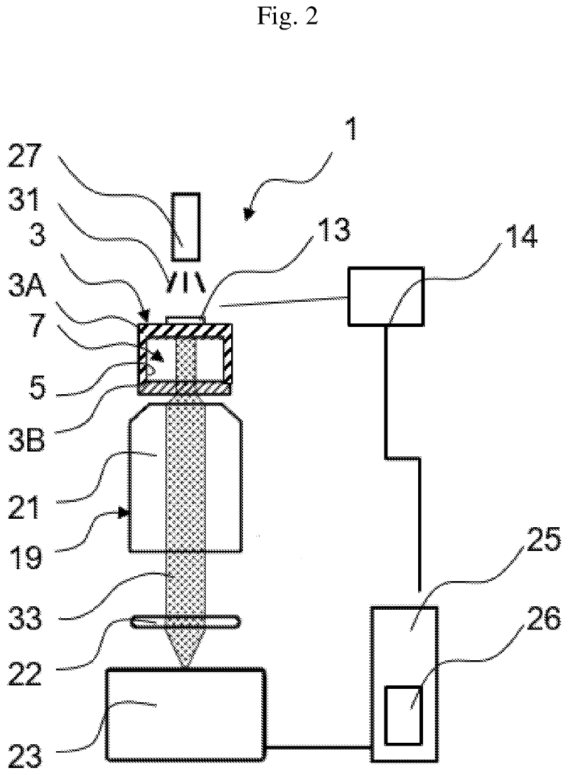

The sample holder 3 comprises a wall 15 providing the holding space 5 with a (functionalized) wall surface portion comprising cells of a first type 17 immobilized on a carrier 15. The shown manipulation system 1 comprises a microscope 19 with an objective 21 and a camera 23 connected with a computer 25 comprising a controller and a memory 26. The computer 25 may also be programmed for tracking one or more of the cells of a second type based on signals from the camera 23 and/or for performing microscopy calculations and/or for performing analysis associated with microscopy and/or video tracking. The computer or another controller (not shown) may be connected with other parts of the system 1 (not shown) for controlling at least part of the microscope 19 and/or another detector (not shown). In particular, the computer 25 may be connected with one or more other parts of the system such as the acoustic wave generator 13, the power supply thereof, the controller 14 thereof (both as shown in FIG. 2), the light source, a temperature control, sample fluid flow control, etc. (none shown).

The system further comprises a light source 27. The light source 27 may illuminate the sample 7 using any suitable optics (not shown) to provide a desired illumination intensity and intensity pattern, e.g. plane wave illumination, Köhler illumination, etc., known per se. Here, in the system light 31 emitted from the light source 27 may be directed through the acoustic wave generator 13 to (the sample 7 in) the sample holder 3 and sample light 33 from the sample 7 is transmitted through the objective 21 and through an optional tube lens 22 and/or further optics (not shown) to the camera 23.

The sample light 33 may comprise light 31 affected by the sample (e.g. scattered and/or absorbed) and/or light emitted by one or more portions of the sample 7 itself e.g. by chromophores and/or fluorophores attached to the cells of a second type 9.

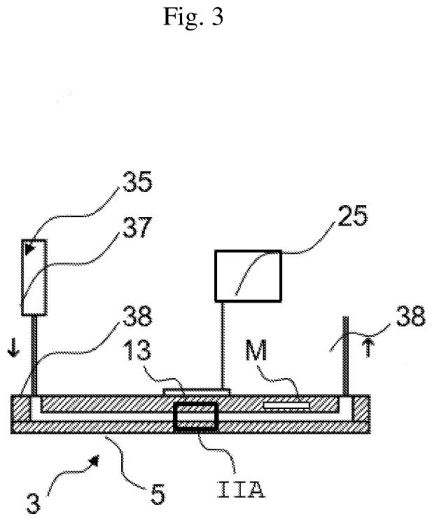

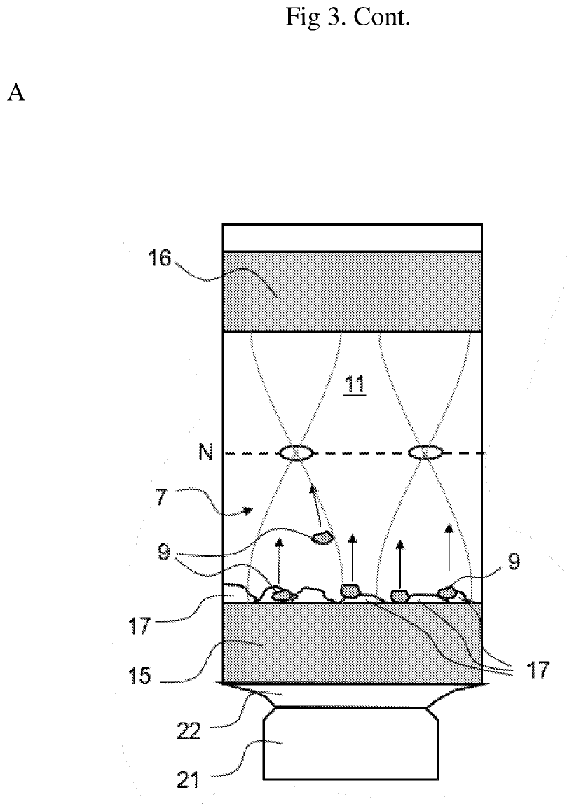

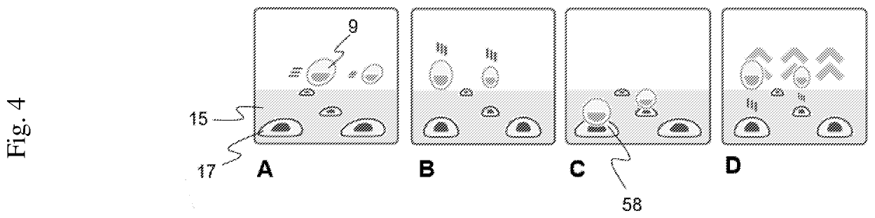

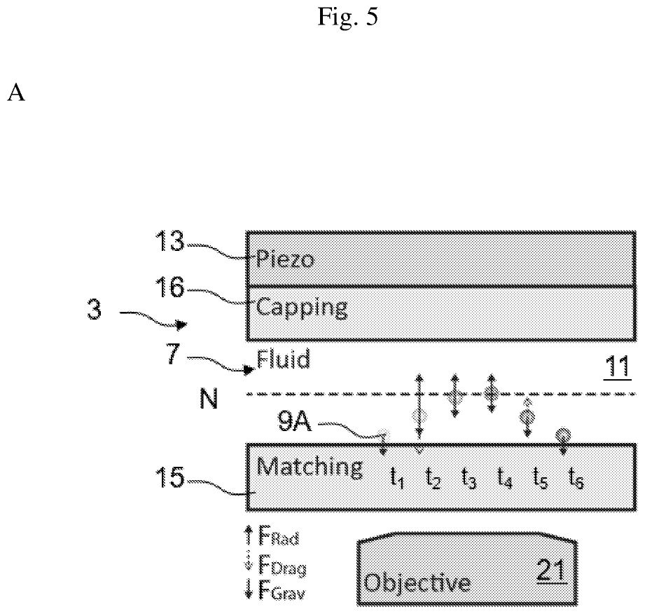

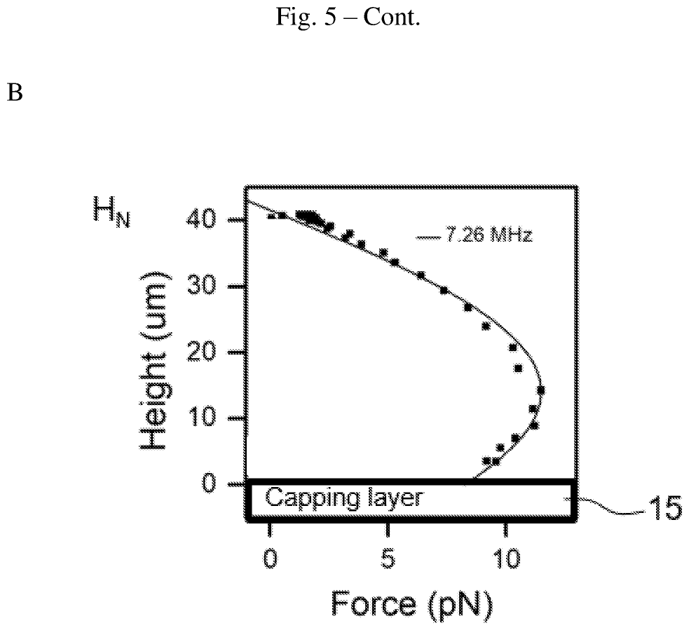

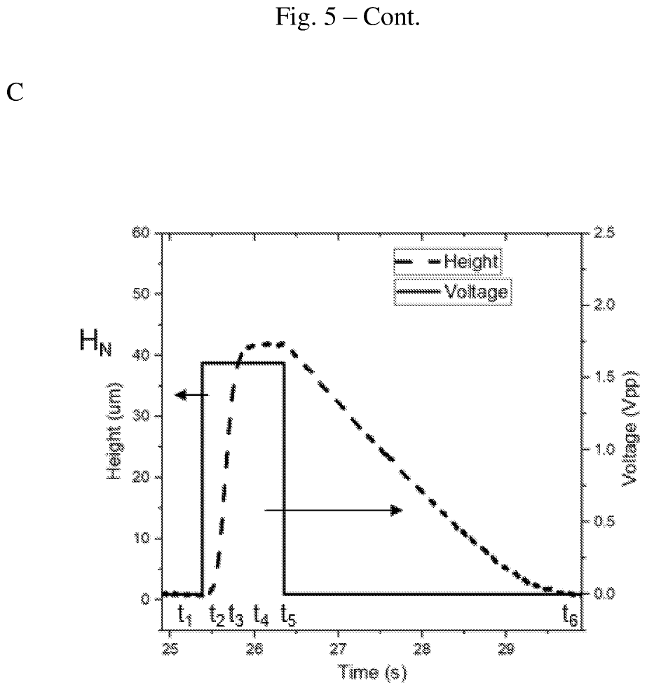

As shown in FIG. 3, the sample holder 3 is connected to an optional fluid flow system 35 for introducing fluid into the holding space 5 of the sample holder 3 and/or removing fluid from the holding space 5, e.g. for flowing fluid through the holding space (see arrows in FIG. 3). The fluid flow system 35 may comprise a manipulation and/or control system, possibly associated with the computer 25. The fluid flow system 35 may comprise one or more of reservoirs 37, pumps, valves, and conduits 38 for introducing and/or removing one or more fluids, sequentially and/or simultaneously. The sample holder 3 and the fluid flow system 35 may comprise connectors, which may be arranged on any suitable location on the sample holder 3, for coupling/decoupling without damaging at least one of the parts 3, 35, and preferable for repeated coupling/decoupling such that one or both parts 3, 35 may be reusable thereafter. FIG. 3A is a schematic of a number of cells of a second type 9 in the sample holder 3 of FIG. 3. Part of the wall 15 of the sample holder 3 is optionally provided with a (functionalized) wall portion comprising cells of a first type 17, e.g. an area of the wall being covered with biological cells of a different type to which the cells of a second type of interest 9 may adhere. Also shown is part of the microscope lens 21 and an optional immersion fluid layer 22 for improving image quality.

On providing a periodic driving signal to the acoustic wave generator 13 a standing wave is generated in the sample holder 3. The signal may be selected, as indicated, such that an antinode of the wave is generated at or close to the wall surface (of the sample holder 3 e.g. surface portion 17) and a node N of the wave W away from the surface 17, generating a local maximum force F on the bodies 9 at or near the surface towards the node. Thus, application of the signal may serve to probe adhesion of the cells of the second type 9 to the cells of the first type 17 in dependence of the force.

In an example an optimal force generation for particular studies may be achieved by selecting acoustic cavity parameters and the frequency/wavelength of the acoustic wave in order to create a maximum pressure gradient at the (functionalised) wall surface, e.g. by ensuring that the distance from the wall surface to the acoustic node is approximately ¼ wavelength.

FIG. 4A-4D: depict schematics of processes occurring in a holding space, e.g. of a microfluidic cell, of an AFS system, wherein the holding space may comprise a (functionalized) wall surface comprising cells of a first type 17 immobilized on a carrier 15 that is configured to bind cells of a second type 9. The holding space may be part of an AFS system as described with reference to FIGS. 2 and 3. The processes in the holding space may be imaged from below or from the top using an imaging system, e.g. as described with reference to FIG. 2. As depicted in FIG. 4A, the process may start with flushing cells of a second type 9, e.g. effector cells, into the holding space, comprising a (functionalized) wall 15 including target cells 17. The introduction of the cells 9 into the holding space may take a predetermined period of time, e.g. between 1 and 5 seconds. After flushing, the cells are allowed to settle onto the (functionalized) wall 15 comprising the target cells 17 (FIG. 4B). When the cells of a second type reach the (functionalized) wall, the cells may move around over the (functionalized) surface until they bind to a location on the surface, for example until they find a suitable target cell to bind to (surveillance) thus forming a bound effector—target cell pair 58 (FIG. 4C). The steps of effector cells settling onto the (functionalized) wall and binding to it may be referred to as the incubation phase. In a typical experiment, incubation may take up to 1-15 minutes or longer. The incubation phase may be imaged and when the cells are introduced into the holding space and move towards the (functionalized) wall, groups of pixels representing cells in the captured images may be detected and tracked. After the incubation phase, a force may be applied to the cells of a second type 9 that are bound to the (functionalized) wall surface. The force may have a direction away from the (functionalized) wall surface, e.g. substantially perpendicular to the (functionalized) wall surface and thus in a direction away from the cells of the first type. Typically, a force ramp will be applied to the cells of a second type, so that if the force becomes larger than a binding force, they will start to move away from the cells of the first type in the direction of the force (FIG. 4D).

When the force is sufficiently large, a cellular body will move away from the (functionalized) wall surface in a direction that depends on the applied force, which may have an axial component perpendicular to the (functionalized) wall (e.g. the z-direction) and two lateral components in the plane of the (functionalized) wall (e.g. the x and y direction). Based on a measurement scheme as described with reference to FIG. 4, various interaction properties between the cells of the first type and the cells of the second type can be determined.

FIGS. 5A-5C: show a method of calibrating an acoustic force in an acoustic force manipulation system. FIG. 5A shows a cross-section of the sample holder including a microscope objective, like FIG. 3A. In the sample fluid 11 a test particle 9A is indicated. FIG. 5A depicts a movement of the particle 9A subject to an acoustic force burst and forces acting on the particle in six points in time t₁ . . . t₆. FIG. 5B shows the acoustic force as a function of height in the holding space between the surface of a wall 15 (“capping”) of the sample holder (height=0) and a node at height H_{N} during the acoustic burst and measured particle positions. FIG. 5C shows the temporal behaviour of the driving signal burst Vpp (right axis) and the height of a particle in a sample holder (left axis).

t₁: no acoustic force is applied and the particle 9A is at rest on the surface of the wall of the sample holder (marked capping) under the influence of the net downward forces of gravity and buoyancy F_{Grav}+F_{buoy};

t₂-t₃: an acoustic wave is generated in the sample holder providing a burst of acoustic force F_{Rad} driving the particle 9A away from the surface towards the node (cf. FIG. 3A). When moving, the particle is now also subject to a drag force F_{Drag} counteracting the movement and F_{Rad}>(F_{Grav}+F_{buoy}). The total force on the particle 9A is Fₜₒₜ=(F_{Grav}+F_{buoy})+F_{Drag}+F_{Rad}>0 (i.e.: upward). Note that the acoustic force F_{Rad} is dependent on the height, see FIG. 5B, and the drag force F_{Drag} is velocity dependent (assuming a spatially and temporally homogeneous sample fluid).

t₄: the acoustic wave and hence the acoustic force is still present, but the particle 9A has come to rest at the node N; F_{Drag}=0 and F_{Rad}=(F_{Grav}+F_{buoy}).

t₅: the driving signal is stopped, the acoustic wave and hence the acoustic force are absent. The particle 9A falls back to the wall 15, counteracted by the drag force F_{Drag}. (F_{Grav}+F_{buoy})>F_{Drag} and Fₜₒₜ=(F_{Grav}+F_{buoy})+F_{Drag}<0 (i.e.: downward)

t₆: no acoustic force is applied and the particle 9A is again at rest on the surface of the wall of the sample holder (marked capping) under the influence of the net downward forces of gravity and buoyancy F_{Grav}+F_{buoy}.

The displacement of the particle 9A may be detected via the microscope using known image capturing techniques including video and/or other time-resolved methods (e.g. methods such as described in WO 2014/200341). The spatiotemporal displacement properties of the particle 9A through the sample fluid may be determined on the basis of the Navier-Stokes equations for the specific particle shape and size in combination with the properties of the fluid 11.

By detecting the displacement velocity of the particle 9A the acoustic force may be determined; note that the same may hold for any lateral displacement of the particle 9A. FIG. 5B shows that an acoustic model (solid line) for a force on the test particle may be fitted to the data. This allows one or more of interpolation, extrapolation and determination of the force at any specific height between the capping layer and the node.

Using a repeating, possibly periodic, driving signal and possibly a small lateral force in one or two directions parallel to the surface and/or perpendicular to the direction of the acoustic force, an acoustic force in plural positions in the holding space distributed in one or two directions perpendicular to the acoustic force direction may be determined.

The acoustic force experienced by a particle in a fluid depends on the size, density and compressibility (acoustic contrast factor) of the particle relative to the fluid. By taking into account the difference in acoustic contrast factor between two particles a force determined for one particle or one number of particles may be used for determining a force for another particle, in particular another particle provided at another time at the same location in the sample holder as the first particle. This may in particular apply if the properties (size, density and compressibility) of one or more (first) particles (e.g. test particles) are known and differ from properties of one or more of the other (second) particles (e.g. cells of a second type). This can be done, for example, by calculating the local (one-, two- or three dimensional) acoustic pressure gradient and/or acoustic energy density based upon the measured force on the one particle or one number of particles. Conversion from force measurement to acoustic energy density and back can for example be done using the following equation taken from Bruus, H. (2012) ‘Acoustofluidics 7: The acoustic radiation force on small particles’, Lab on a Chip, The Royal Society of Chemistry, pp. 1014-1021. doi: 10.1039/c2lc21068a:

F_{z}^{rad}=−∂_{z}U^{rad}=4πφ_{P}(ρ_{P}, ρ_{f}, β_{P}, β_{f})ka³E_{ac}sin(2kz)

here F_{z}^{rad} is the acoustic radiation force, U^{rad} is the acoustic radiation force potential, φ_{P} is the acoustic contrast factor for the particle, β_{P} is the compressibility of the particle, β_{f} is the compressibility of the fluid, ρ_{P} is the density of the particle, ρ_{f} is the density of the fluid, k is the wavenumber, a is the particle radius, E_{ac} is the acoustic energy density, and z is the distance from the wall. The acoustic contrast factor φ_{P}(ρ_{P}, ρ_{f}, β_{P}, β_{f}) of a particle may be defined as follows:

$${φ}_{P} = \frac{5 {ρ}_{B} - 2 {ρ}_{f}}{2 {ρ}_{B} + {ρ}_{f}} - \frac{{β}_{p}}{{β}_{f}}$$

The acoustic contrast factor defines how the acoustic wave interacts with the cellular body. In case the contrast factor is larger than 0, a cellular body will move towards an acoustic node, if it is smaller than 0 it will move away from the acoustic node.

### DEFINITIONS

A portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

As used herein, the singular form terms “a,” “an,” and “the” include plural referents unless the content clearly dictates otherwise. Thus, for example, reference to “a cell” includes a combination of two or more cells, and the like.

As used herein, “about” and “approximately”, when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed invention.

As used herein, “and/or” refers to a situation wherein one or more of the stated cases may occur, alone or in combination with at least one of the stated cases, up to with all of the stated cases.

As used herein, “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. As used herein, the term “at most” a particular value means that particular value or less. For example, “at most 5” is understood to be the same as “5 or less” i.e., 5, 4, 3, . . . , −10, −11, etc.

As used herein, “comprising” or “to comprise” is construed as being inclusive and open ended, and not exclusive. Specifically, the term and variations thereof mean the specified features, steps or components are included. These terms are not to be interpreted to exclude the presence of other features, steps or components. It also encompasses the more limiting “consisting” or “to consist of”.

As used herein, “conventional techniques” or “methods known to the skilled person” refer to a situation wherein the methods of carrying out the conventional techniques used in methods of the invention will be evident to the skilled worker. The practice of conventional techniques in molecular biology, biochemistry, cell culture, genomics, sequencing, medical treatment, pharmacology, immunology and related fields are well-known to those of skill in the art and are discussed, in various handbooks and literature references.

As used herein, “exemplary” or “for example” means “serving as an example, instance, or illustration,” and should not be construed as excluding other configurations, including those disclosed herein.

As used herein, “antagonist” and/or “inhibitor” are used interchangeably, and they refer to a compound or agent having the ability to reduce or inhibit a biological function of a target protein or polypeptide, such as by reducing or inhibiting the activity or expression of the target protein or polypeptide. Accordingly, the terms “antagonist” and “inhibitor” are defined in the context of the biological role of the target protein or polypeptide. While some antagonists herein specifically interact with (e.g., bind to) the target, compounds that inhibit a biological activity of the target protein or polypeptide by interacting with other members of the signal transduction pathway of which the target protein or polypeptide are also specifically included within this definition. According to the current invention, the antagonist is an antagonist of LFA-1. In some embodiments the antagonist of LFA-1 is a compound that has the ability to reduces or inhibit the interaction of LFA-1 with one of its (natural) ligands. LFA-1 has 6 known ligands: ICAM-1, ICAM-2, ICAM-3, ICAM-4, ICAM-5, and JAM-A (see, for example Ley, K (2007) Adhesion molecules: function and inhibition; ISBN 9783764379759).

### DETAILED DESCRIPTION

It is contemplated that any method, use or composition described herein can be implemented with respect to any other method, use or composition described herein. Embodiments discussed in the context of methods, use and/or compositions of the invention may be employed with respect to any other method, use or composition described herein. Thus, an embodiment pertaining to one method, use or composition may be applied to other methods, uses and compositions of the invention as well.

Techniques for studying interactions between cells are well known, some examples have been described in the art: the article by Hosseini et al. PNAS 2009 106:42 17852-17857 doi“10.1073”pnas.0905384106 is an example of the use of Atomic Force Microscopy (AFM) to probe the binding between cells. The article by Zhang et al. Science Translational Medicine 2016, 8(341), 341ra77 1-341ra77 8, doi 10.1126/scitranslmed.aaf1278 does a similar probing of interaction strengths using micropipettes. Some of these techniques are sometimes also referred to as force spectroscopy techniques.

Another example of such technique and methodology is disclosed in detail in WO2018/083193. This patent application describes a so-called acoustic force spectroscopy (AFS) system that is configured to examine interactions between cells by applying force to the cells. The system includes a microfluidic cell comprising a carrier in the form of a wall surface that may have cells of a first type, for example target cells expressing antigens, immobilized thereupon. A plurality of cells of a second type, for example lymphocytes such as T cells, can be introduced into the microfluidic cell comprising the immobilized cells of the first type. The cells of the first type and the cells of the second type may than interact with each other and bind with each other. Thereafter, an acoustic source is used to exert a force on the cells (e.g. a ramping force) such that the cells of the second type, for example the T cells, may detach from the cells of the first type, for example the target cells expressing an antigen, at a certain force. During this process, the spatiotemporal behavior of the cells of the second type may be monitored using an imaging microscope. The interaction between the cells of the first type and the cells of the second type and, for example the force at which the cells of the second type may detach from the cells of the first type, may be determined by analyzing the captured video images. For example, the cell avidity of the effector cells can be determined this way.

During the process cells, in particular cells of the second type, may be sorted and collected, for example based on the force at which the cells of the second type detach from the cells of the first type. In a preferred embodiment of the current invention the technique as described above is used.

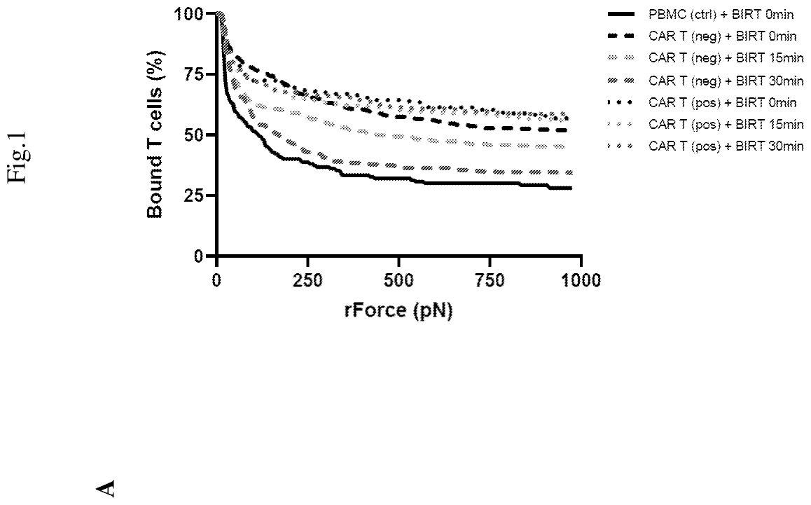

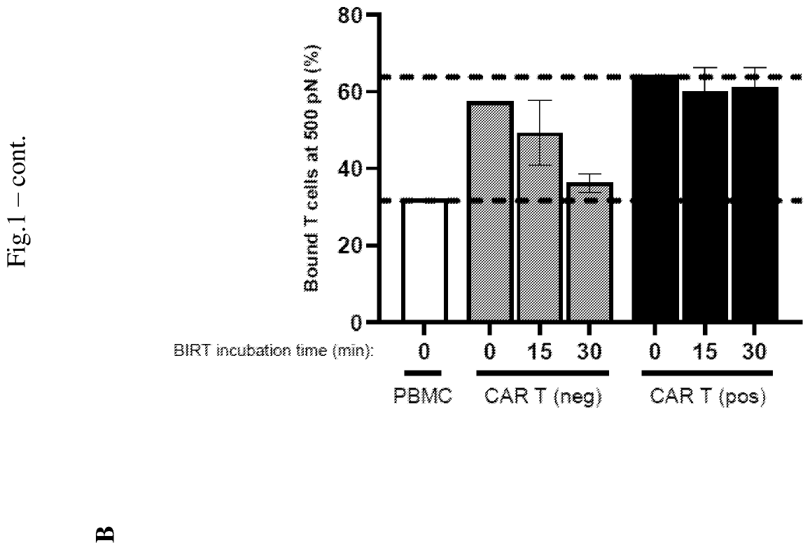

As can be witnessed from the Examples and Figures provided herein, it was found that when studying the interaction between and binding of target cells expressing an antigen with CAR T cells, there is still substantial interaction and binding observed when CAR T cells are used that express CARs that do not specifically bind to an antigen expressed on the immobilized target cells (“specifically binds” refers to the binding comprising specific antigen-binding receptor interaction, for example specific binding between an antigen presented on an APC and a TCR). As can be witnessed from, for example, FIG. 1A, this may make it more difficult to differentiate between, in this example, CAR T cells that specifically bind (i.e. via CAR-antigen interaction) and CAR T cells that should not specifically bind with the immobilized target cells.

As the skilled person will understand, such non-specific binding of, in this example, CAR T cells to the target cells will therefore reduce the signal-to-noise ratio and makes it more difficult to study, measure, identify or obtain, in this example, CAR T cells that express a CAR that does specifically bind to an antigen on the surface of the target cells. This will in particular be true in mixed populations of, for example, T cells expressing different type of (unidentified) TCRs, and of which only few T cells will express a TCR that may specifically bind to the antigen expressed by the target cells.

Based on the above observation the inventors of the current invention therefor set-out to provide for an improved method that would better allow to differentiate between the binding of lymphocytes to target cells expressing an antigen, in particular to better differentiate between specific and non-specific binding of lymphocytes to target cells expressing an antigen.

Surprisingly it was found that it is possible to provide for a solution by using, in the method, an antagonist of the LFA-1 protein that is expressed on the cell surface of lymphocytes. As can be witnessed from the Examples, by using an antagonist of LFA-1 the percentage of non-specific binding of lymphocytes to target cells expressing an antigen is dramatically reduced, even to the level of what is observed when using non-lymphocytic control cells (in the example peripheral blood mononuclear cells (PBMCs) are used).

In other words, when studying interaction between and binding of lymphocytes to target cells a high level of non-specific binding is observed, the level of non-specific binding being substantially higher than that observed when using non-lymphocytic control cells (in the example peripheral blood mononuclear cells (PBMCs). This suggests strong interactions between lymphocytes and target cells, which interactions are independent of the specific binding of the lymphocytes to an antigen on the target cells. Such interactions may for example relate to what is referred to in the prior art as kinapses, and/or may for example be related to the process of antigen-receptor recognition. By using an antagonist of LFA-1 this non-specific binding of the lymphocytes to the target cells is dramatically reduced while, surprisingly, as shown in the Examples, the specific interaction between lymphocytes and target cells is hardly or not affected by the presence of the LFA-1 antagonist.

Accordingly, the invention is as defined in the accompanying claims.

Accordingly, there is provided for a method of binding lymphocytes to target cells, the method comprising
a) providing cells of a first type and providing cells of a second type, wherein the cells of the first type are immobilized on a carrier;
b) contacting the cells of the first type with the cells of the second type in order to allow the cells of the first type and the cells of the second type to bind to each other;
c) applying a force to the cells of the second type, wherein the force is in a direction away from the cells of the first type;

and wherein

A: the cells of the first type are target cells that express at least one antigen and the cells of the second type are lymphocytes; or

B: the cells of the first type are lymphocytes and the cells of the second type are target cells that express at least one antigen;

and wherein

the cells of the first type, the cells of the second type, or the cells of the first type and the cells of the second type are contacted with an antagonist of LFA-1 before, during, and/or after contacting the cells of the first type with the cells of the second type.

With the method of the invention lymphocytes are allowed to bind to target cells. In the method cells of a first type are provided. The cells of the first type are immobilized to a carrier.

As will be understood by the skilled person the carrier to which the cells are immobilized is not in any particular way limited and may be of any suitable material, including for example glass, plastics, matrices (e.g. extracellular matrices such as Matrigel), polymers and that are suitable and/or regularly used in the field for the attachment of cells thereto. It will also be understood by the skilled person that such carriers may be coated with suitable materials (for example poly-L-lysine) to improve the attachment of the cells of the first type to the carrier. It will be understood by the skilled person that, in the context of the invention, the carrier to which the cells of the first type are immobilized serves the purpose of allowing the application of a force onto the cells of the second type so that, as the consequence of that force, the interaction between the cells of the first type and the cells of the second type may, at a certain force, be broken (“ruptured”), so that the cells of the second type may detach from the cells of the first type and move away from the cells of the first type that are (and remain) immobilized on the carrier. The carrier may, for example, be in the form of beads, but may also be in the form of a glass slide or may form the wall of a reactor vessel in which the method according to the invention is performed.

It will also be understood that while a force is being applied to the second cells, a force may also act on the first cells. This may be hard to avoid depending on the method of force application (e.g. when acoustic forces are applied relative forces acting on the first and second cells may depend on the respective acoustic contrast factor of the first and second cells and when a centrifugal force is applied relative forces acting on the first and second cells may depend on the respective density difference of the first and second cells with respect to the medium). As long as the first cells are appropriately and sufficiently bound to the carrier they may remain attached to the carrier during the force application and the binding force between the first and second cells may be effectively probed.

As will be understood by the skilled person the force applied to the cells is a force that is “pulling away” from the adhesion force (i.e. the force that defines the strength of binding between cells) that may exist between a cell of a first type and a cell of the second type. Such force may in the field of force spectroscopy also be referred to as a rupture force (see also Kamsma et al (2018). Cell Reports, 24(11), 3008-3016. https://doi.org/10.1016/j.celrep.2018.08.034).

As will be understood, within the context of the current invention such rupture force is preferably non-disruptive with respect to the integrity of the cells, i.e. the force does not damage or destroy the cells but may disrupt the interaction or binding between the cells of the first type and the cells of the second type. //nlp

Depending on the configuration of the method, the cells of the first type may be the lymphocytes or may be the target cells that express at least one antigen.

Also provided in the method of the invention are cells of a second type. The cells of the second type may or may not be of the same type as the cells of the first type (for example, the cells of the first type are lymphocytes expressing an antigen and the cells of the second type are the same lymphocytes). Preferably the cells of the second type are distinct and/or different from the cells of the first type. In contrast to the cells of the first type, the cells of the second type are not immobilized on a carrier, and are for example comprised in a suitable aqueous medium that allows the cells of the second type to free move and/or settle. Depending on the configuration of the method, the cells of the first type may be the lymphocytes or may the target cells that express at least one antigen. Thus, in some embodiments the cells of the first type are target cells that express at least one antigen and the cells of the second type are lymphocytes. Thus, in some embodiments the cells of the first type are lymphocytes and the cells of the second type are target cells that express at least one antigen.

It will be appreciated by the skilled person that the cells of the first type or the cells of the second type do not need to be identical cells. In other words, the cells of the first type and the cells of the second type may, independently, consist of cells that are distinct from each other and may not consist of homogeneous cell populations, or cells of the same type (e.g. lymphocytes or cancer cells). For example, in case the cells are target cells, the cells may each express different antigens (i.e. a first target cell expressing a first antigen and a second target cells expressing a second antigen that is different from the first antigen). For example, in case the cells are lymphocytes, the lymphocytes, for example T cells, may consist of a population of lymphocytes that may differ from each other in the expression of different TCRs on the cells surface of the cells (for example, a first T cell may express a first (unidentified) TCR and a second T cell may express a second (unidentified) TCR and wherein the first and second TCR may be different).

It will also be understood by the skilled person that the number of cells of either the first type or the second type that are provided is not in any particular way limited by the method of the invention.

In a next step of the method of the invention, the cells of the first type and the cells of the second type are brought into contact with each other. The cells of the first type and the cells of the second type are contacted with each other under conditions that allow the cells of the first type and the cells of the second type to interact and bind to each other. Although not in particular limited thereto, in the practice of the current invention, and in a preferred embodiment, the cells of the second type are introduced into, for example, the reactor vessel that already comprises the immobilized cells of the first type, in a suitable aqueous medium. The skilled person understands, within the context of the current invention, what conditions are suitable to allow the cells of the second type to interact and bind to the cells of the first type. For example, the skilled person understand what type of aqueous medium and what type of temperature can suitably be used in the method of the invention. For example, in some preferred embodiments, steps b) and c) of the method of the invention are preferably performed at a temperature between 0 degrees Celsius and 40 degrees Celsius, for example at a temperature between 0 degrees Celsius and 10 degrees Celsius or at a temperature between 35 degrees Celsius and 38 degrees Celsius, for example at about 0, 1, 2, . . . , 10, . . . , 20, . . . , 35, 36, 37, 38 or 39 degrees Celsius.

Contacting of the cells of the second type with the cells of the first type may or may not be performed under gentle stirring or shaking of the (aqueous) medium that comprises the cells of the first type and the cells of the second type.

As will be understood by the skilled person, also contemplated is that the cells of the first type and the cells of the second type are contacted with each other before the cells of the first type are immobilized on the carrier. Also in such case cells of a first type and cells of a second type are contacted with each other, and the cells of the first type are immobilized on a carrier (in this case, after the cells of the first type and the cells of the second type are contacted with each other). In such specific embodiment, covered by the broader embodiments described herein, one would thus be providing cells of a first type and providing cells of a second type, contacting the cells of the first type with the cells of the second type in order to allow the cells of the first type and the cells of the second type to bind to each other, and wherein the cells of the first type are immobilized on a carrier.

In even a further embodiment of the method of the invention, the cells of the first type and the cells of the second type are allowed to contact each other before the cells of the first type are immobilized on a carrier, and wherein further the cells of the second type may also be immobilized on a (different) carrier. For example both the cells of the first type and the cells of the second type may be immobilized on (different) beads, after which a force is applied on the (different) bead (for example using optical tweezers) in order to probe rupture forces. Obviously in such embodiment the cells of the first type may be immobilized on such beads, and the cells of the second type may be immobilized on beads before the cells are contacted with each other.

After the cells of the first type and the cells of the second type are contacted with each other in step b), in step c) a force is applied to the cells of the second type, wherein the force is in a direction away from the cells of the first type. As already explained above, and as will be appreciated by the skilled person the force applied to the cells is a force that is “pulling away” from the adhesion force (i.e. the force that defines the strength of binding between cells) that may exist between a cell of a first type and a cell of the second type. In other words, the force applied to the cells pulls on the cells of the second type is such way that these cells, when not attached or bound to the cells of the first type, move in a direction away from the cells of the first type (whereas the cells of the first type will remain immobilized on the carrier), thereby separating the cells of the first type from the cells of the second type that are not bound. The cells of the second type that remain bound to the cells of the first type and, for example, did not detach as the consequence of the applied force, cannot freely move in a direction away from the cell of the first type, and are therefore also separated from the cells of the second type but that are not bound. In other words, the applied force may, for a given, interacting, pair of a cell of the first type and a cell of the second type be such that the force causes the detachment of the cell of the second type from the cell of the first type, for example because the force is such that the interaction between the two cells is ruptured (the force pulling on the cell of the second type is larger than the adhesion force between the cell of the first type and the cell of the second type).

As mentioned before, preferably the force is such that it does not damage or destroy the cells, thus allowing the retrieval of the (living) cells for further use.

The skilled person will understand that in case the force applied to the cells is not strong enough to detach the cells of the second type from the cells of the first type, he may increase the force applied. This will allow the gradual or stepwise detachment of the cells of the second type from the immobilized cells of the first type. Within the context of the current invention, the skilled person will be able to determine what forces should be applied to the cells of the second type such that these may detach from the cells of the first type and move away from these cells of the first type. The skilled person will understand that the strength of the force will depend on, for example, the type of the force applied, the type of target cells and the type of lymphocytes used and the experimental conditions (e.g. temperature, presence of test compounds, condition of the cells etc.) and can establish this without undue burden.

It will thus be understood that by applying a force to the cells of the second type, the strength of the binding of the cells of the second type to the cells of the first type may be monitored, studied, observed or determined. It will also be understood that by applying a force or by applying a force with varying strength over time, cells of the second type that are not bound or that detach from the cells of the first type may be separated from cells of the second type that are (or remain) bound to the cells of the first type. It will thus be understood that cells may be separated from each other based on the strength of the applied force and obtained for further use and analysis.

As already mentioned above, and as shown in the Examples, it is an important feature of the current invention that the cells of the first type, the cells of the second type, or the cells of the first type and the cells of the second type are contacted with an antagonist of LFA-1 before, during, and/or after contacting the cells of the first type with the cells of the second type. It was surprisingly found that by contacting the cells of the first type, the cells of the second type, or the cells of the first type and the cells of the second type with an antagonist of LFA-1, non-specific binding of the lymphocytes to the target cells can dramatically be reduced, thereby allowing the improved detection, measurement or obtaining of lymphocytes that, in the method of the invention, engage in specific, or at least stronger, binding with the target cells.

The cells of the first type and/or the cells of the second type may be contacted with the antagonist of LFA-1 before, during, and/or after the cells of the first type and the cells of the second type are contacted with each other. For example, the cells of the first type may be contacted with the antagonist of LFA-1 before the cells of the second type are added. Alternative, or in addition, the cells of the second type may be treated with the antagonist of LFA-1 before they are added to the cells of the first type. Indeed, it is also contemplated that both the cells of the first type and the cells of the second type are each, independently, contacted with an antagonist of LFA-1 before the cells are contacted with each other. It will be appreciated that, preferably, when the cells of the first type and/or the cells of the second type are contacted with the antagonist of LFA-1 before they are contacted with each other, this is performed in a (relatively short) time period directly before the cells are contacted with each other, as will be detailed herein elsewhere.

It is also contemplated that when the cells of the first type and the cells of the second type are contacted with each other, at the same time they are contacted with the antagonist of LFA-1. Also contemplated is that first the cells of the first type and the cells of the second type are contacted with each other in the absence of an antagonist of LFA-1 and that there after an antagonist of LFA-1 is added.

In some embodiments the cells of the first type and/or the cells of the second type are contacted with the antagonist of LFA-1 before the cells are contacted with each other. Preferably the antagonist of LFA-1 is also present when the cells of the first type and the cells of the second type are contacted with each other.

It is also contemplated that more than one antagonist of LFA-1 is used in the method of the invention, either separate, during the different stages as described above (before, during or after contacting of the cells of the first type with the cells of the second type), or as a mixture. For example, it is contemplated that in the method of the invention a first antagonist of LFA-1 is used that specifically binds with LFA-1 and therewith interferes with the interaction of LFA-1 with one of its ligands, and a second antagonist of LFA-1 is used that specifically binds with one of the ligands (e.g. ICAM-1) and therewith interferes with the interaction of LFA-1 with the ligand.

It will be understood by the skilled person that the concentration of the antagonist of LFA-1 to be used in the method of the current invention will depend on the type of the antagonist of LFA-1 to be used. However, in view of the current disclosure, the skilled person is well suited to determine such suitable concentration of an antagonist of LFA-1 by simple experimentation, for example as set out in the Examples provided herein. By testing varying concentrations of the particular antagonist of LFA-1, it can be determined in a straight-forward manner, and without undue experimentation, what concentration of the antagonist of LFA-1 may suitably be used in the method of the invention.

Without being bound by theory, it is believed by the inventors, that with the method according to the invention in particular the formation or maintenance of kinapses between the lymphocytes and the target cells may be prevented, inhibited or reduced whereas the formation or maintenance of (mature) synapses between the lymphocytes and the target cells remains unaffected, or at most only disturbed to a lesser extent. Target cells and lymphocytes that are not able to form (mature) synapses or that are only able to form relatively weaker synapses, but that are still able to form kinapses may therefore now surprisingly be distinguished from those target cells and lymphocytes that specifically bind to each other (and form stable immunological synapses).

Also provided is for the method according to the invention wherein the force that is applied to the cells of the second type introduces a (pulling) force onto the cells of the second type so that at least part of the cells of the second type may detach from the cells of the first type and move away from the cells of the first type.

As already described herein elsewhere, and as will be understood by the skilled person, the force to be applied to the cells of the second type is a force that is in a direction away from the cells of the first type. In other words, the force introduces a pulling force onto the cells of the second type so that in a certain force range (at a certain strength of the force) the cells of the first type may detach from the cells of the first type and that are immobilized on the carrier.

Because the cells of the first type are immobilized on a carrier, and when a force is applied to the cells in a direction away from the cells of the first type, or alternatively said, in a direction away from the carrier on which the cell of the first type are attached, such force will force the cells of the second type to be pulled into a direction away from the carrier/from the cells of the first type immobilized on the carrier. If the force applied is strong enough the interaction between the cells of the first type and the cells of the second type may be disrupted (“ruptured”), causing the cells of the second type to detach from the cells of the first type. While the cells of the first type will remain immobilized on the carrier and cannot move away from the carrier, the cells of the second type that detached can now, under the influence of the applied force, move away (can be pulled away) from the cells of the first type that are immobilized on the carrier.

The skilled person understand how to immobilize the cell of the first type is a suitable manner. For example the skilled person may use a carrier comprising a substrate known to allow cells to immobilize (e.g. polylysine, collagen, gelatin, poly-L-ornithine, vitronectin, fibronectin, and so). Suitable substrates and or coating for immobilizing the cell of the first type are well-known to the skilled person. Such substrate may be useful to resist the pulling force of, for example, the acoustic waves. In such using a carrier comprising a substrate (such as polylysine) can allow forces that pull the cells apart, without affecting the monolayer cells.

Also provided is for the method according to the invention wherein the contacting of the cells of the first type with the cells of the second type in step b) is for an incubation period of between 1 second and 60 minutes. It was found that the method of the invention allows a long incubation period wherein the cells of the first type and the cells of the second type are contacted with each other. As will be understood by the skilled person, depending on, for example the temperature, the concentration of the antagonist of LFA-1, or the type of lymphocyte and/or target cell used in the method of the invention, the incubation period during which the cells of the first type and the cells of the second type are contacted with each other may be varied. However, in a preferred embodiment the incubation step of step b) of the method of the invention is for a period of between 1 second and 60 minutes, for example, between 1 second and 30 minutes, between 1 second and 15 minutes, between 5 seconds and 15 minutes, or between 10 seconds and 10 minutes.

Also provided is for the method according to the invention wherein

i) the lymphocytes are first contacted with the antagonist of LFA-1 for an incubation period T1 before the lymphocytes are contacted with the target cells in step b), preferably in the presence of the antagonist of LFA-1;

ii) the target cells are first contacted with an antagonist of LFA-1 for an incubation period T1 before the target cells are contacted with the lymphocytes in step b), preferably in the presence of the antagonist of LFA-1;

iii) the cells of the first type and the cells of the second type are contacted with each other in step b) in the absence of an antagonist of LFA-1 for an incubation period T2 and subsequently in the presence of an antagonist of LFA-1 for an incubation period T3; or

iv) the cells of the first type and the cells of the second type are contacted with each other in step b) in the presence of an antagonist of LFA-1 for an incubation period T4.

According to the method of the invention the cells of the first type, the cells of the second type, or the cells of the first type and the cells of the second type are contacted with an antagonist of LFA-1 before, during, and/or after contacting the cells of the first type with the cells of the second type cells. Independently of whether the lymphocytes used in the method of the invention are the cells of the first type or the cells of the second type, in a preferred embodiment the lymphocytes are first contacted with the antagonist of LFA-1 for an incubation period T1 before the lymphocytes are contacted with the target cells in step b), preferably in the presence of the antagonist of LFA-1. Likewise, independently of whether the target cells used in the method of the invention are the cells of the first type or the cells of the second type, in a preferred embodiment the target cells are first contacted with the antagonist of LFA-1 for an incubation period T1 before the target cells are contacted with the lymphocytes in step b), preferably in the presence of the antagonist of LFA-1. In these embodiments, the lymphocytes, the target cells, or both, are, independently contacted with the antagonist of LFA-1 for an incubation period T1. After the incubation period T1, the lymphocytes are contacted with the target cells in step b) of the method according to the invention. It is contemplated that in these embodiments, the cells are contacted with each other in step b) in the absence of the antagonist of LFA-1, however, preferably the cells are contacted with each other during step b) in the presence of the antagonist of LFA-1.

In a preferred embodiment, incubation period T1 is between 1 second and 180 minutes, for example between 1 second and 60 minutes, or between 1 minute and 60 minutes, or between 5 minutes and 60 minutes or between 5 minutes and 30 minutes. As can be witnessed from the Examples, with an increasing incubation period T1, in the presence of an antagonist of LFA-1, non-specific binding between lymphocytes and target cells is reduced and thus improves observing, measuring, and/or separating lymphocytes that specifically or more strongly bind to the target cells.

In another embodiment of the method according to the invention, the cells of the first type and the cells of the second type are contacted with each other in step b) in the absence of an antagonist of LFA-1 for an incubation period T2 and subsequently in the presence of an antagonist of LFA-1 for an incubation period T3. In such embodiment of the method of the invention the cells of the first type and the cells of the second type are contacted with each other for an incubation period T2 in the absence of an antagonist of LFA-1. After the incubation period T2, the antagonist of LFA-1 is provided to the cells for an incubation period T3. In a preferred embodiment incubation period T2 is between 1 second and 60 minutes and incubation period T3 is between 1 second and 60 minutes. Preferably incubation period T2 and incubation period T3 are together no more than 120 minutes, preferably no more than 60 minutes. Preferably incubation period T2 is between 30 seconds and 30 minutes, for example between 1 minute and 10 minutes, or between 30 seconds and 5 minutes. Preferably incubation period T3 is between 1 second and 60 minutes, for example between 1 minute and 60 minutes, for example between 5 minutes and 60 minutes.

Also contemplated is an embodiment in which the cells of the first type and the cells of the second type are contacted with each other in step b) in the presence of an antagonist of LFA-1 for an incubation period T4. In this embodiment the lymphocytes and the target cells are (first) contacted with each other in the presence of an antagonist of LFA-1. In this embodiment the cells of the first type and the cells of the second type may or may not, independently of each other, have been contacted with the antagonist of LFA-1 before the cells were contacted with each other (in the presence of the antagonist of LFA-1. The incubation period T4 is, as already mentioned above, preferably between 1 second and 60 minutes, for example between 1 minute and 60 minutes, for example between 5 minutes and 60 minutes.

Also provided is for the method according to the invention wherein the antagonist of LFA-1 is selected from the group consisting of BIRT-377, an LFA-1 binding antibody, an ICAM-1 binding antibody, an LFA-1 antagonist, an ICAM-1 antagonist.

As described herein, the antagonist of LFA-1 is a compound or agent having the ability to reduce or inhibit a biological function of LFA-1. LFA-1 (Lymphocyte function-associated antigen-1) is a well-known member of the integrin superfamily. LFA-1 is composed of α- and β-subunits that together form a heterodimer expressed at the cell surface. These subunits include long extracellular domains, a single transmembrane domain, and short cytoplasmic tails (Walling et al. Front Immunol. 2018; 9: 952; doi: 10.3389/fimmu.2018.00952). The expression of LFA-1 is believed to be restricted to leukocytes including lymphocytes (such as T-cells, B-cells, and NK cells), neutrophils, monocytes, macrophages, dendritic cells, mast cells, and eosinophils and is overexpressed in certain lymphomas and leukemias. The major ligands of LFA-1 identified to date belong to the immunoglobulin (Ig) superfamily. They include the intercellular adhesion molecules ICAM-1, ICAM-2, ICAM-3, ICAM-4, ICAM-5 and the junctional adhesion molecule JAM-A (previously JAM-1). These ligands are expressed on various cell types including endothelial cells lining the vessel wall, epithelial and tissue resident cells (e.g. keratinocytes, dendritic cells) leukocytes and antigen presenting cells. LFA-1 is believed to play a central role in the innate and adaptive immune response and is crucial for the activation of immune cells. In this context, LFA-1 is, for example, well-characterized as a co-stimulatory receptor which is essential for the formation of the immunological synapse and controls T cell activation and proliferation. Normally, LFA-1 resides on the cell surface in an inactive state. Upon intracellular signaling (so-called “inside-out” signaling) LFA-1 is converted from an inactive to an active, ligand-binding state. This conversion is associated with major conformational changes within the receptor. Upon ligand binding, LFA-1 conveys signals back into the cells (so-called “outside-in” signaling), triggering subsequent steps which depend on the cell type. LFA-1 is, for example, a key T cell integrin, which plays a major role in regulating T cell activation and migration. Adhesion to LFA-1's ligand, in particular intracellular adhesion receptor 1 (ICAM-1) facilitates firm endothelium adhesion, prolonged contact with antigen-presenting cells, and binding to target cells for killing.

The antagonist of LFA-1 is a compound or agent that has the ability to reduce or inhibit a biological function of LFA-1. In a preferred embodiment the antagonist of LFA-1 is a compound or agent that has the ability to reduce or inhibit binding of LFA-1 with one of its ligands. In a preferred embodiment the antagonist of LFA-1 is a compound or agent that has the ability to reduce or inhibit binding of LFA-1 with ICAM-1. ICAM-1 (Intercellular Adhesion Molecule 1) also known as CD54 (Cluster of Differentiation 54) antigen is a cell surface glycoprotein which is typically expressed on endothelial cells and cells of the immune system. ICAM-1 is an endothelial- and leukocyte-associated transmembrane protein well-known for its importance in stabilizing cell-cell interactions and facilitating leukocyte endothelial transmigration (Walling et al. Front Immunol. 2018; 9: 952; doi: 10.3389/fimmu.2018.00952).

In a preferred embodiment the antagonist of LFA-1 is a compound or agent that has the ability to reduce or inhibit a biological function of LFA-1 by binding to LFA-1. In a preferred embodiment the antagonist of LFA-1 is a compound or agent that has the ability to reduce or inhibit a biological function of LFA-1 by binding to a ligand of LFA-1, preferably by binding to ICAM-1. In a preferred embodiment, the antagonist of LFA-1 is a small molecule or an antibody. In an embodiment the antibody is an antibody that binds to LFA-1 and/or an antibody that binds to ICAM-1. In some embodiments more than one antagonist of LFA-1 is used simultaneously in the method of the invention.

The skilled person is well aware of antagonists of LFA-1 and various have been described in the prior art (reviewed in Giblin et al. Curr Pharm Des. 2006; 12(22):2771-95. doi: 10.2174/138161206777947731 and Kapp et al. Expert Opin Ther Pat. 2013 October; 23(10):1273-95). Non-limiting examples include compounds disclosed in WO2015189265, WO0059878, and WO2004073701. Other non-limiting examples include the pharmaceutical drug lifitegrast which inhibits LFA-1 from binding to intercellular adhesion molecule 1 (ICAM-1), SAR 1118 (Paskowitz et al. Investigative Ophthalmology & Visual Science April 2011, Vol.52, 570.) and other compounds or agents known to inhibit, in particular, adhesion molecule-1 (ICAM-1)/leukocyte function-associated antigen-1 (LFA-1) interaction (e.g. SAR 1118 Zhong et al., ACS Med. Chem. Lett., 2012, 3(3):203; Several LFA-1 inhibiting antibodies (7E4, TS1/18, M HM23, TS1/22, M HM24) Gronholm et al., Blood, 2016, 128(9)).

In a particular preferred embodiment according to the invention the antagonist of LFA-1 is BIRT-377 (Kelly et al., J. Immunol., 1999, 163:1573; Woska et al. J Leukoc Biol. 2001 August; 70(2):329-34. doi.org/10.1189/jIb.70.2.329). BIRT-377 ((5R)-5-[(4-Bromophenyl) methyl]-3-(3, 5-dichlorophenyl)-1,5-dimethyl-2,4-imidazolidinedione) is a potent negative allosteric (reversible) modulator of LFA-1 and is commercially available, for example from Tocris (www.tocris.com/products/birt-377_4776).

As discussed above, the skilled person can easily establish suitable concentrations of the antagonist of LFA-1 for use in the method of the invention. For example, the skilled person may test various concentrations of a particular antagonist of LFA-1 in the method of the invention and determine a (range of) concentrations of the antagonist of LFA-1 most suitably used in a particular experiment. The skilled person understands that such concentration may, for example, depend on the type of lymphocytes and/or target cells used, as well as the experimental conditions under which the method is performed. For example, a non-limiting concentration of BIRT-377 that may be used in the method according to the invention may vary between 0.1 and 35 microgram per milliliter, for example between 0.5 and 20 microgram per milliliter, for example between 1 and 10 microgram per milliliter.

Also provided is for the method according to the invention wherein the lymphocytes are selected from the group consisting of T cells, NK cells, B cells, CAR T cells, CAR NK cells, dual CAR T cells, dual CAR NK cells, multiple CAR T cells, multiple CAR NK cells, tandem CAR T cells, tandem CAR NK cells, transgenic TCR T cells, transgenic TCR NK cells, T-cells decorated with T-cell engagers, and NK-cells decorated with NK-cell engagers. Preferably the lymphocytes are T cells.

The term T cell also includes Natural Killer T-cells (Godfrey et al. Nature Reviews Immunology volume 4, pages231-237(2004)). As discussed herein, the current invention is based on the use of an antagonist of LFA-1. LFA-1 is expressed in leucocytes, including lymphocytes, including T cells, B cells (Carrasco et al Immunity. 2004 May;20(5):589-99. doi: 10.1016/s1074-7613(04)00105-0.) and NK cells (Urlaub et al Immunol. 2017 Mar 1;198(5):1944-1951. doi: 10.4049/jimmuno1.1601004), and all these cells can interact with target cells via interaction between LFA-1 and a ligand of LFA-1 on the target cell, in particular ICAM-1. Modified cells obtained from these lymphocytes such as CAR T cells, CAR NK cells, dual CAR T cells, dual CAR NK cells, multiple CAR T cells, multiple CAR NK cells, tandem CAR T cells, tandem CAR NK cells, transgenic TCR T cells, transgenic TCR NK cells, T cells with T cell engagers, and NK cells with NK-cell engagers likewise express LFA-1 and may interact with target cells by interaction between LFA-1 and a ligand of LFA-1, in particular ICAM-1 expressed on the cell surface of the target cell (see, for example, Kantari-Mimoun et al doi.org/10.1101/2020.05.27.119198, or, Morrelo et al. doi.org/10.1016/j.jtho.2016.11.405).

The skilled person is well aware of the different type of lymphocytes, including those described herein. Chimeric antigen receptor T cells (CAR T cells) are T cells that have been genetically engineered to express an artificial T cell receptor, the chimeric antigen receptors; CAR NK cells are NK cells (natural killer cells) that also express such chimeric antigen receptors. Such CAR NK cells are being developed for (potential) use in, for example, immunotherapy. Such NK cells engineered to express CARs have potential benefits compared to CAR T cells. NK cells have spontaneous cytotoxic activity and can generate target cell death independent of tumor antigen, while CAR T lymphocytes only kill their targets by a CAR specific mechanism. Therefore, in the setting of antigen downregulation by tumor cells attempting to escape immune detection, NK cells would still be effective against tumor cells (Habib et al. Ochsner J. 2019 Fall; 19(3): 186-187. doi: Dual CAR or multiple CAR T cells or NK cells are T cells or NK cells that express two or multiple different CAR's in the same cell while tandem CAR T cells or tandem CAR NK cells are cells that encode two (or more) CARs on the same chimeric protein using a single vector (see, for example, Shah et al Front Oncol. 2019; 9: 146. doi: 10.3389/fonc.2019.00146). Transgenic TCR T cells and transgenic TCR NK cells are cells that have been provided with genes encoding for a specific T cell receptor, and express such transgenic TCR on the cell surface (see, for example, Ivanov et al Clin Exp Immunol. 2006 Jan; 143(1): 78-84. doi: 10.1111/j.1365-2249.2005.02967.x or Mensali et al EBioMedicine. 2019; 40:106-117. doi: 10.1016/j.ebiom.2019.01.031. Epub 2019 Jan 18.). T cells with T cell engagers, and NK cells with NK cell engagers are cells that have associated with it so-called bispecific or multi-specific T cell engagers or NK cell engagers. These engagers, for example so-called BiTE's, target a cell surface protein expressed on the T cell or NK cell and at the same time target antigens present on target cells (for example tumor cells). By binding to antigens on the target cell and binding to a cell surface protein on the T cell or NK cell such engagers, including BiTEs may mediate T cell or NK cell responses and, may, for example enhance killing of tumor cells (see, for example, Slaney et al . Cancer Discovery 2018:924 dot: 10.1158/2159-8290.CD-18-0297).

The skilled person understands that in the method according to the invention one specific type of lymphocyte may be used, but that it is also possible to use different lymphocytes in the same method. For example, it is contemplated that two or more different CAR-T cells, each expressing a different chimeric antigen receptor, are used in the method of the invention. It is also contemplated that, for example T cells and NK cells are used together in the method of the invention.

The target cell used in the method of the invention may be any cell that expresses at least one antigen, including antigen presenting cells (APC), tumor cells, dendritic cells, macrophage, B cell, primary cells, cell lines and the like. In the context of the current invention an antigen is to be understood to be any molecule, polypeptide, polysaccharide or protein that can presented on the surface of the target cell and with which a lymphocyte may interact by specific binding to such antigen. The antigen may be complexed with major histocompatibility complexes, but also be presented on the surface of a cell, for example of a cancer cell.

Also provided is for the method according to the invention wherein the lymphocytes and the target cells are human cells. The cells may have been directly obtained from a subject, but may also be of a cell line. The lymphocytes and the target cells used in the method according to the invention may however originate from a wide variety of other vertebrates such as, for example from mammals, fish, rodents, rabbits, primates, monkeys and the like. Although it is contemplated that in the method of the invention lymphocytes and target cells may be used that each originate from a different organism (for example lymphocytes that originate from human and target cells that originate from rabbit), in a preferred embodiment the lymphocytes and the target cells originate from the same organism (e.g. both originate from human). In another embodiment the lymphocytes and the target cells both originate from the same subject (for example from a patient). In a preferred embodiment the lymphocytes and the target cells are human cells.

Also provided is for the method according to the invention wherein the cells of the first type are target cells that express at least one antigen and the cells of the second type are lymphocytes, preferably T cells (which, by definition includes different types of T cells including CAR T cells). With this preferred embodiment of the method according to the invention, the cells of the second type are the lymphocytes, which may be studied for their binding to the target cells and may be sorted and obtained for further use (for example for identification of one or more specific antigen-binding receptors expressed by the sorted and obtained lymphocytes and/or for expansion and infusion and/or use in therapy).

Also provided is for the method according to the invention wherein the cells of the first type are immobilized on a carrier that forms a layer or wall, preferably wherein the layer is a wall in a sample holder in which the cells of the first type are contacted with the cells of the second type. Although the type of (inert) carrier on which the cells of the first type are immobilized is not in any particular way limited, and may for example be formed by a bead, a pearl, a wafer, a membrane and the like, in a preferred embodiment the carrier is in the form of a layer on which multiple cells of the first type can be immobilized. The cells that are immobilized on such layer may form, for example, a monolayer, preferably a monolayer with a high confluency. In a particular preferred embodiment, the layer on which the cells of the first type can be immobilized forms a wall in or of a reactor vessel in which the method according to the invention may be performed.

Also provided is for the method according to the invention wherein the cells of the first type are contacted with the cells of the second type in the presence of candidate compounds suspected to modulate binding of the cells of the first type and the cells of the second type. As described herein, the method of the invention allows for the improved detection, monitoring and/or separation of lymphocytes and/or target cells that specifically, or stronger, bind or interact with each other. It is thereof also contemplated that the method of the invention can be used to identify, screen or test compounds or agents that may influence such interaction between the lymphocyte and the target cell. In other words, also provided is for a method according to the invention wherein the lymphocytes and the target cells are contacted with each other in the presence of one or more candidate compounds that are suspected to modulate binding, preferably specific binding, of the lymphocytes to the target cells. The skilled person appreciates that by comparing to the situation wherein the one or more candidate compounds are not present, compounds may be identified that either enhance or diminish the interaction of the lymphocytes with the target cells under the conditions studied. Such compounds may be identified as candidate drug compounds in the development of therapies that would benefit from enhanced interaction between the lymphocytes and the target cells or that would benefit from diminished interaction between the lymphocytes and the target cells. With the method of the invention it has become possible to quickly and reliably screen for such compounds.

Also provided is for the method according to the invention wherein the method further comprises during or after step c),
collecting the cells of the second type that are not bound to or that detached from the cells of the first type and, optionally, collecting the cells of the first and/or second type that remain bound;
detecting the cells of the second type that are not bound to the cells of the first type and/or detecting the cells of the second type that are bound to the cells of the first type, preferably wherein detecting comprises optical detection, imaging, cell sorting, cell counting, and/or force measurements; and/or
determining interaction between the cells of the first type and the cells of the second type, wherein determining interaction comprises optical detection, imaging, cell sorting, cell counting, and/or force measurements.
   The skilled person is well-aware of methods of optical detection, imaging, cell sorting, cell counting, and/or force measurements that can suitably be used in the method according to the invention.

Also provided is for the method according to the invention wherein the force applied to the cells of the second type is selected from the group consisting of a force exerted by generating an acoustic wave, a centrifugal force, and a magnetic force. Technique for studying interactions between cells may be referred to as force spectroscopy techniques and are well-known to the skilled person (see for example, the review “A practical review on the measurement tools for cellular adhesion force” by Ungai-Salánki et al in Advances in Colloid and Interface Science (2019) 269: 309-333; doi.org/10.1016/j.cis.2019.05.005). Such techniques include the use of a force exerted by generating an acoustic wave, a centrifugal force, and a magnetic force.

As an example of the use of acoustic waves to generate the force reference is made to for example WO2018/083193 (as discussed herein elsewhere) and to z-Movi, the cell avidity analyzer developed by Lumicks (Amsterdam, The Netherlands; lumicks.com/products/z-movi-cell-interaction-studies/).

For example, the cells of the second type may labeled with (a) magnetic bead(s) and by using a magnetic force in direction away from the cells of the first type (or directed away from the carrier on which these cells of the first type are immobilized) the binding interaction between the cells can be disrupted (raptured) and/or be analyzed or measured (see, for example, Ino et al. Biotechnology and Bioengineering 102:3 pp 882-890; doi.org/10.1002/bit.22104).

As will be understood by the skilled person, within the method of the invention one strength of the applied force may be used. However, in an alternative embodiment, the strength of the force applied to the lymphocytes is varied over time, preferably wherein the strength of the force is increased over time, preferably wherein the force is a force exerted by generation of an acoustic wave, and wherein the force is varied over time, for example wherein the real force applied is up to 5000 pN (e.g. using the z-Movi device as referred to herein).

As discussed herein elsewhere, the skilled person understands how to apply a force to the cells of the second type within the context of the current invention. At the same time the skilled person understands or can easily determine the strength of the force that is to be applied. As will be understood, by increasing the applied force over time, cells of the second type that did not detach initially may detach at increasing force strength, and can thus analyzed, studied, observed or collected separately from those cells that already detached initially and/or from those cells that still remain bound to the cells of the first type.

Finally, there is provided for a method according to the invention wherein the force applied in step c) is applied for a period of between 1 second and 60 minutes. Preferably the force is applied for a period of between 1 second and 30 minutes, for examples less than 20, 10 or 5 minutes.

Having now generally described the invention, it will be understood by the skilled person that all details, embodiments and preferences discussed with respect to one aspect of an embodiment of the invention is likewise applicable to any other aspect or embodiment of the invention and that there is therefore not need to detail all such details, embodiments and preferences for all aspect separately.

Having now generally described the invention, the same will be more readily understood through reference to the following examples which is provided by way of illustration and is not intended to be limiting of the present invention.

### EXAMPLES

### Methods

Target cells (50×10e6/mL) were seeded on the glass surface of a z-Movi® chip that was pretreated with poly-L-lysine (0.002%) for 15 minutes at 37 degrees Celsius. The target cells were allowed to adhere to the glass surface, and form a monolayer, for 2 hours in RPMI-1640 culture medium without serum. After the 2-hour incubation period the monolayer cells were supplemented with RPMI-1640 culture medium containing 10% serum, and ready for use.

Primary human T cells were generated to express a control non-specific (neg) or specific (pos) chimeric antigen receptor (CAR) following viral transduction. In addition, non-transduced peripheral blood mononuclear cells (PBMCs) were used for additional negative controls and CAR-independent background binding.

Before use primary human T cells and PBMCs were stained with CellTrace™ Far Red dye (C34564, Invitrogen) for easy tracking. Prior to co-culture with monolayer cells labeled CAR expressing T cells were incubated with BIRT-377 (10 ug/mL) or vehicle control (DMSO) for indicated time (i.e. 0, 15 or 30 minutes). After incubation, T cells and PBMCs (both 10×10e6/mL) were subsequently co-cultured with the monolayer cells for 5 minutes and analyzed for specific binding to these monolayer cells in a temperature-controlled (37 degrees Celsius) z-Movi chip using a z-Movi instrument by applying an increasing acoustic force ramp (0-1000 pN, in 2.5 minutes) to the interacting cells (for example, in line with what is disclosed in Kamsma et al. Cell Reports, 24(11), 3008-3016. https://doi.org/10.1016/j.celrep.2018.08.034 or Fernandez de Larrea; doi: 10.1158/2643-3230.BCD-20-0020).

### Results

As can be witnessed from FIG. 1. The data shows that the avidity for the target monolayer of the specific CAR expressing T cells (CAR T (pos)) is the highest. Importantly, the avidity curves demonstrate that increasing the incubation time with BIRT-377 shifts the non-specific CAR T cells (CAR T (neg)) towards the background control (PBMCs) (FIG. 1A). In contrast, when specific CAR T cells were preincubated with BIRT-377 the effect on the avidity curve is marginal (FIG. 1A). Moreover, when looking at the plateau phase of the PBMC control curve, setting in at 500 pN, the data reveals that the percentage of non-specific interacting T cells (CAR T (neg)) decreases in a BIRT-377 incubation time-dependent manner, reaching nearly a similar percentage as the untreated PBMCs (FIG. 1B), as indicated by the lower dotted line.

On the other hand, treatment with BIRT-377 does not affect the specific binding of CAR T cells (pos) to the monolayer, as indicated by the upper dotted line.

Together, these data reveal that BIRT-377 can reduce antigen-independent interactions (e.g. kinapses; see Dustin et al. Immunol Rev. 2008 Feb; 221:77-89. doi: 10.1111/j.1600-065X.2008.00589.x.) as demonstrated for CAR T (neg) cells, while maintaining the specific interactions (immunological synapses) shown by CAR T (pos) cells. As such, this allows for better separation of cells that specifically bind to their target monolayer cells in an antigen-specific (antigen-dependent) manner.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

All references cited herein, including journal articles or abstracts, published or corresponding patent applications, patents, or any other references, are entirely incorporated by reference herein, including all data, tables, figures, supporting information and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

1. A method of binding lymphocytes to target cells, the method comprising:
(a) providing cells of a first type and providing cells of a second type, wherein the cells of the first type are immobilized on a carrier;
(b) contacting the cells of the first type with the cells of the second type in order to allow the cells of the first type and the cells of the second type to bind to each other;
(c) applying a force to the cells of the second type, wherein the force is in a direction away from the cells of the first type;
and wherein
A: the cells of the first type are target cells that express at least one antigen and the cells of the second type are lymphocytes; or
B: the cells of the first type are lymphocytes and the cells of the second type are target cells that express at least one antigen;
and wherein
the cells of the first type, the cells of the second type, or the cells of the first type and the cells of the second type are contacted with an antagonist of LFA-1 before, during, and/or after contacting the cells of the first type with the cells of the second type.

2. The method of claim 1 wherein the force that is applied to the cells of the second type introduces a force onto the cells of the second type so that at least part of the cells of the second type may detach from the cells of the first type and move away from the cells of the first type.

3. The method of claim 1 wherein the contacting of the cells of the first type with the cells of the second type in step b) is for an incubation period of between 1 second and 60 minutes.

4. The method of claim 1 wherein
i) the lymphocytes are first contacted with the antagonist of LFA-1 for an incubation period T1 before the lymphocytes are contacted with the target cells in step b), preferably in the presence of the antagonist of LFA-1;
ii) the target cells are first contacted with an antagonist of LFA-1 for an incubation period T1 before the target cells are contacted with the lymphocytes in step b), preferably in the presence of the antagonist of LFA-1;
iii) the cells of the first type and the cells of the second type are contacted with each other in step b) in the absence of an antagonist of LFA-1 for an incubation period T2 and subsequently in the presence of an antagonist of LFA-1 for an incubation period T3; or
iv) the cells of the first type and the cells of the second type are contacted with each other in step b) in the presence of an antagonist of LFA-1 for an incubation period T4.

5. The method according to claim 4 wherein the incubation period T1 is between 1 second and 180 minutes, the incubation period T2 is for between 1 second and 60 minutes, the incubation period T3 is for between 1 second and 60 minutes, and/or the incubation period T4 is for between 1 second and 60 minutes.

6. The method according to claim 1 wherein the antagonist of LFA-1 is selected from the group consisting of BIRT-377, an LFA-1 binding antibody, and an ICAM-1 binding antibody.

7. The method according to claim 1 wherein the lymphocytes are selected from the group consisting of T cells, NK cells, B cells, CAR T cells, CAR NK cells, dual CAR T cells, dual CAR NK cells, multiple CAR T cells, multiple CAR NK cells, tandem CAR T cells, tandem CAR NK cells, transgenic TCR T cells, transgenic TCR NK cells, T-cells decorated with T-cell engagers, and NK-cells decorated with NK-cell engagers.

8. The method according to claim 1 wherein the cells of the first type and or the cells of the second type are human cells.

9. The method according to claim 1 wherein the cells of the first type are target cells that express at least one antigen and the cells of the second type are lymphocytes.

10. The method according to claim 1 wherein the cells of the first type are immobilized on a carrier that forms a layer or wall, preferably wherein the layer is a wall in a sample holder in which the cells of the first type are contacted with the cells of the second type.

11. The method according to claim 1 wherein the cells of the first type are contacted with the cells of the second type in the presence of candidate compounds suspected to modulate binding of the cells of the first type and the cells of the second type.

12. The method according to claim 1 wherein the method further comprises during or after step c)
collecting the cells of the second type that are not bound to or that detached from the cells of the first type and, optionally, collecting the cells of the first and/or second type that remain bound;
detecting the cells of the second type that are not bound to the cells of the first type and/or detecting the cells of the second type that are bound to the cells of the first type, preferably wherein detecting comprises optical detection, imaging, cell sorting, cell counting, and/or force measurements; and/or
determining interaction between the cells of the first type and the cells of the second type, wherein determining interaction comprises optical detection, imaging, cell sorting, cell counting, and/or force measurements.

13. The method according to claim 1 wherein the force applied to the cells of the second type is selected from the group consisting of a force exerted by generating an acoustic wave, a centrifugal force, and a magnetic force.

14. The method according to claim 1 wherein the strength of the force applied to the cells of the second type is varied over time, preferably wherein the strength of the force is increased over time, preferably wherein the force is a force exerted by generation of an acoustic wave, and wherein the force is varied over time.

15. The method according to claim 1 wherein the force applied in step c) is applied for a period of between 1 second and 60 minutes.
